(19) United States
(12) Patent Application Publication
(10) Pub. No.: US 2024/0033410 A1
(43) Pub. Date: Feb. 1, 2024

(54) OXYGENATOR, AND EXTRACORPOREAL MEMBRANE OXYGENATION DEVICE

(71) Applicants: BEIHANG UNIVERSITY, Beijing (CN); BEIJING AEROSPACE CHANGFENG CO., LTD., Beijing (CN)

(72) Inventors: Zengsheng CHEN, Beijing (CN); Mingzhou XU, Beijing (CN); Yubo FAN, Beijing (CN); Zihua SU, Beijing (CN); Yawei WANG, Beijing (CN); Minghao YUE, Beijing (CN)

(21) Appl. No.: 18/266,112

(22) PCT Filed: Sep. 19, 2022
(86) PCT No.: PCT/CN2022/119495

§ 371 Date: Jun. 8, 2023

(30) Foreign Application Priority Data
Jul. 19, 2021 (CN) 202110814717.X

(51) Int. Cl. A61M 1/16

(52) U.S. Cl. CPC A61M 1/1698; A61M 1/1629

(57) ABSTRACT

Disclosed are an oxygenator and an extracorporeal membrane oxygenation device. The oxygenator includes: a housing, an upper end cover of the housing being provided with a blood inlet, and a lower end cover of the housing being provided with a blood outlet; and an oxygenation chamber, arranged in the housing, where the axis of the blood inlet and the axis of the blood outlet coincide with the axis of the oxygenation chamber. The blood inlet and the blood outlet are arranged at the center of the upper and lower parts of the oxygenation chamber, after the blood entering the oxygenation chamber is uniformly diffused to the periphery, the blood uniformly flows to the blood outlet from top to bottom due to gravity. The extracorporeal membrane oxygenation device includes the described oxygenator.

### TECHNICAL FIELD

The present disclosure relates to the field of medical devices, and in particular to an oxygenator, and an extracorporeal membrane oxygenation device.

### BACKGROUND

Extracorporeal membrane oxygenation (ECMO) represents an advanced technology in the field of extracorporeal circulation equipment, while oxygenator (membrane oxygenator) is the key device in ECMO system, with the main function of blood-oxygen exchange and carbon dioxide removal. In the research and design of the membrane oxygenator, the design of blood flow path, gas path and heat exchange water path is particularly important, which directly affects gas-blood exchange performance, blood compatibility and heat transfer performance of the membrane oxygenator. For example, if the blood flow and gas paths are not designed well, many flow dead zones may exist in the membrane oxygenator, and the resistance of blood passing through the membrane oxygenator is large, and thus the damage to the blood flowing through membrane fibers is increased, the probability of thrombosis is increased, the gas-blood exchange efficiency is seriously affected.

At present, although ECMO used in clinic practice has treated many patients, there are still problems, such as high incidence of thrombus, low gas exchange efficiency, and poor biocompatibility. This is directly related to non-uniform flow field and pressure field inside the membrane oxygenator, the existence of flow dead zones, slow flow velocity and long-term blood retention. The occurrence of thrombus may directly affect the function of the membrane oxygenator, reduce the gas exchange efficiency, and even increase the risk of thrombosis.

### SUMMARY

An objective of the present disclosure is to provide an oxygenator, and an extracorporeal membrane oxygenation device using the oxygenator, through which a flow field and a pressure field in an oxygenation chamber can be uniformly distributed, a flow dead zone or a flow disturbance zone can be eliminated, thereby reducing the probability of thrombosis.

In a first aspect of embodiments of the present disclosure, an oxygenator is provided, including:
a housing, where an upper end cover of the housing is provided with a blood inlet, and a lower end cover of the housing is provided with a blood outlet; and
an oxygenation chamber, arranged in the housing, where the axis of the blood inlet and the axis of the blood outlet coincide with the axis of the oxygenation chamber.

In this embodiment, as the blood inlet and the blood outlet are arranged at the center of the upper and lower parts of the oxygenation chamber, after the blood uniformly diffused to the periphery in an inlet buffer zone, the blood uniformly flows to the blood outlet from top to bottom due to gravity. The blood flow resistance is low, the gas-blood exchange is sufficient, and the blood retention time is short. Moreover, during the blood flow, flow fields and pressure fields in the oxygenation chamber are uniformly distributed, flow dead zones or flow disturbance zones can be eliminated, thereby reducing the probability of thrombosis.

In some embodiments, an interval space is formed between the upper end cover of the housing and an upper end face of the oxygenation chamber, and an interval space is formed between the lower end cover of the housing and a lower end face of the oxygenation chamber. Spaces of the blood inlet and the blood outlet are gradually reduced in directions away from the interval spaces, respectively.

In this embodiment, the interval space located on the upper end face of the oxygenation chamber can make the blood diffused prior to entering the oxygenation chamber without the interference of a gas-blood exchange module, the diffusion effect is better, and the diffusion efficiency is higher. The interval space located on the lower end face of the oxygenation chamber is used to lengthen a circulation path of the blood, such that the blood can flow through this interval space before flowing out of the blood outlet, and possible dead zones are set outside the oxygenation chamber, thereby further reducing the probability of thrombosis.

In some embodiments, an inner surface of the lower end cover of the housing is a tapered face, and the blood outlet is provided at the lowest point of the tapered face.

In this embodiment, the inner surface of the lower end cover of the housing is designed as a tapered face, which can pool the blood from the periphery of the interval space to the blood outlet, thus preventing the flow dead zone from occurring in the interval space to affect the blood circulation performance.

In some embodiments, the upper end face and lower end face of the oxygenation chamber are respectively provided with a first orifice plate and a second orifice plate for communicating the oxygenation chamber with the interval spaces, respectively.

In this embodiment, the first orifice plate and the second orifice plate not only can be used to fix a gas-blood exchange module in the oxygenation chamber, but also can improve the blood diffusion effect through holes uniformly provided on the orifice plates. In addition, the second orifice plate may also be used to drain the blood at the periphery in the oxygenation chamber, so as to reduce the occurrence of the dead zone.

In some embodiments, a first gas exchange cavity and a second gas exchange cavity are formed between a side face of the oxygenation chamber and a side face of the housing, and are used to exchange gas with the outside of the housing.

In this embodiment, the first gas exchange cavity and the second gas exchange cavity can directly carry out gas-blood exchange with the blood inside the oxygenation chamber without additionally providing a gas-blood exchange module, and thus the system structure can be simplified.

In some embodiments, multiple hollow permeable tubes are arranged in the oxygenation chamber in a horizontal direction. One end of each of the hollow permeable tubes communicates with the first gas exchange cavity, and the other end of the hollow permeable tube communicates with the second gas exchange cavity.

In this embodiment, the hollow permeable tubes are arranged in the oxygenation chamber, and are used to carry out gas-blood exchange with the blood in the oxygenation chamber. Although a gas-blood exchange module is additionally provided, the hollow permeable tubes can be uniformly distributed inside the oxygenation chamber, and better gas-blood exchange efficiency can be achieved.

In some embodiments, the multiple hollow permeable tubes are arranged in a layered and crossed manner.

In this embodiment, the hollow permeable tubes are horizontally arranged in a layered and crossed manner, such that a gas-blood exchange path is divided into two paths from one. With the characteristics that the blood is diffused to the periphery in this embodiment, the gas-blood exchange efficiency of membrane fibers can be greatly improved, and thus the efficacy of a membrane oxygenator is further improved.

In some embodiments, the oxygenator further includes:
a heat exchange chamber arranged in the housing, where a lower end face of the heat exchange chamber is connected to the upper end face of the oxygenation chamber, and the axis of the heat exchange chamber coincides with the axis of the oxygenation chamber.

In this embodiment, the oxygenator is additionally provided with the heat exchange chamber on the basis of the foregoing embodiments, thus making the oxygenator integrate a heat exchange function without changing the original performance.

In some embodiments, an upper end face of the heat exchange chamber is provided with a third orifice plate.

In this embodiment, the third orifice plate is used for communicating the heat exchange chamber with the blood inlet or the interval space, and also has a function of improving the diffusion effect of the blood.

In some embodiments, a first heat exchange cavity and a second heat exchange cavity are provided between a side face of the heat exchange chamber and the side face of the housing, and are connected to the outside of the housing for heat exchange.

In this embodiment, the first heat exchange cavity and the second exchange cavity may directly exchange heat with blood in the heat exchange chamber without additionally providing a gas-heat exchange module, and thus the system structure can be simplified.

In some embodiments, multiple heat exchange tubes are arranged in the heat exchange chamber in a horizontal direction. One end of each of the heat exchange tubes communicates with the first heat exchange cavity, and the other end of the heat exchange tube communicates with the second heat exchange cavity.

In this embodiment, the heat exchange tubes are arranged in the heat exchange chamber, and the heat exchange tubes are used to exchange heat with the blood in the heat exchange chamber. Although a heat exchange module is additionally provided, the heat exchange tubes can be uniformly distributed inside the heat exchange chamber, and better heat exchange efficiency can be achieved.

In some embodiments, the multiple heat exchange tubes are arranged in a layered and crossed manner.

In this embodiment, as the heat exchange tubes are horizontally arranged in a layered and crossed manner, a heat exchange path can be divided into two paths from one. With the characteristics that the blood is diffused to the periphery in this embodiment, the heat exchange efficiency can be greatly improved, and thus the efficacy of the membrane oxygenator is improved.

In a second aspect of the embodiments of the present disclosure, an extracorporeal membrane oxygenation device is provided, including the oxygenator of any one of the foregoing embodiments.

The embodiments of the present disclosure have the following beneficial technical effects:

In accordance with the embodiments of the present disclosure, the blood inlet and the blood outlet are arranged at the center of the upper and lower part of the oxygenation chamber, such that after the blood entering the oxygenation chamber is uniformly diffused to the periphery, the blood uniformly flows to the blood outlet from top to bottom due to gravity. The flow fields and the pressure fields in the oxygenation chamber are uniformly distributed, the blood flow resistance is low and the blood retention time is short, and the flow dead zones or the flow disturbance zones can be eliminated, thereby reducing the probability of thrombosis.

### BRIEF DESCRIPTION OF THE DRAWINGS

FIG. 1A is a sectional view of an oxygenator according to embodiments of the present disclosure;

FIG. 1B is a partial enlarged view of a blood outlet of an oxygenator according to embodiments of the present disclosure;

FIG. 1C is a top view of an oxygenator according to embodiments of the present disclosure;

FIG. 1D is a bottom view of an oxygenator according to embodiments of the present disclosure;

FIG. 2 is a design schematic diagram of a gas path of an oxygenator according to embodiments of the present disclosure;

FIG. 3A is a sectional view of an oxygenator according to embodiments of the present disclosure;

FIG. 3B is a bottom view of an oxygenator according to embodiments of the present disclosure;

FIG. 4 is a design schematic diagram of a water flow path of an oxygenator according to embodiments of the present disclosure.

In the drawings:
1—housing, 101—blood inlet, 102—blood outlet, 103—gas hole, 104—gas hole, 105—first blood sampling port, 106—temperature measurement port, 107—second blood sampling port, 108—first exhaust port, 109—second exhaust port, 110—heat exchange hole, 111—heat exchange hole, 112—upper end cover, 113—lower end cover, 2—oxygenation chamber, 201—upper end face, 202—lower end face, 3—interval space, 4—interval space, 5—hollow permeable tube, 6—first orifice plate, 7—second orifice plate, 8—mounting block, 9—connector, 10—first gas exchange chamber, 11—second gas exchange chamber, 12—heat exchange chamber, 13—third orifice plate, 14—heat exchange tube, 15—first heat exchange cavity, 16—second heat exchange cavity.

### DETAILED DESCRIPTION OF THE EMBODIMENTS

To make the objectives, features and advantages of the present disclosure more clearly, the following further describes the present disclosure in detail with reference to the specific embodiments and the accompanying drawings. It should be understood that these descriptions are only exemplary, and are not intended to limit the scope of the present disclosure. In addition, in the following description, descriptions of well-known structures and technologies are omitted to avoid unnecessarily confusing the concepts of the present disclosure.

The applicant found that although ECMO used in clinic practice has treated many patients, there are still many problems, such as high incidence of thrombus, low gas-blood exchange efficiency, and poor biocompatibility. This is directly related to non-uniform flow fields and pressure fields inside the membrane oxygenator, the existence of flow dead zones, slow flow velocity and long-term blood retention. The occurrence of thrombus may directly affect the function of the membrane oxygenator, reduce the gas exchange efficiency, and even increase the risk of thrombosis.

Based on reasons above, the applicant found in the research that by optimizing the design of a blood flow path, a gas path and a water path of the membranous oxygenator, flow fields and pressure fields inside the membrane oxygenator can be uniformly distributed, a flow retention zone is small, the blood flow resistance is low, and the gas-blood exchange efficiency and the heat exchange efficiency are high, thus improving the efficacy of long-term support of the membrane oxygenator and reducing the probability of thrombosis during long-term support.

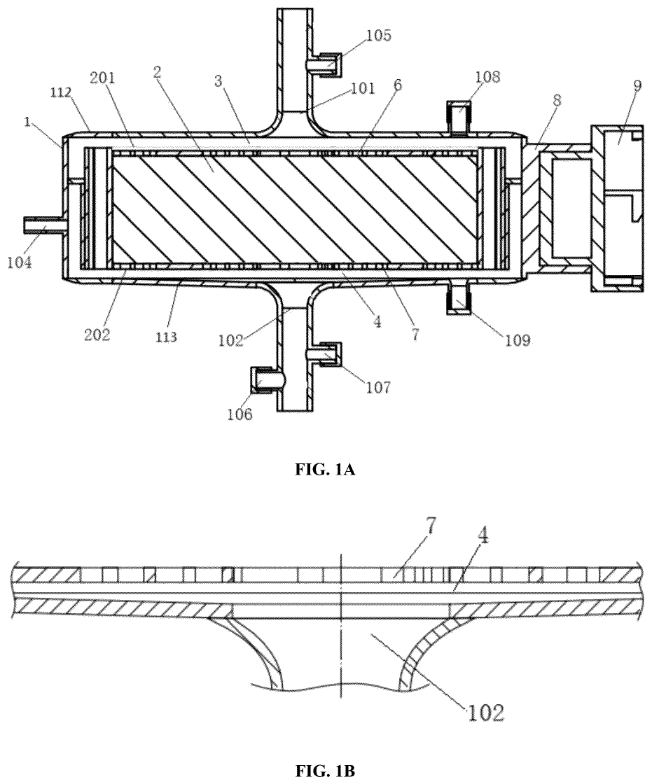

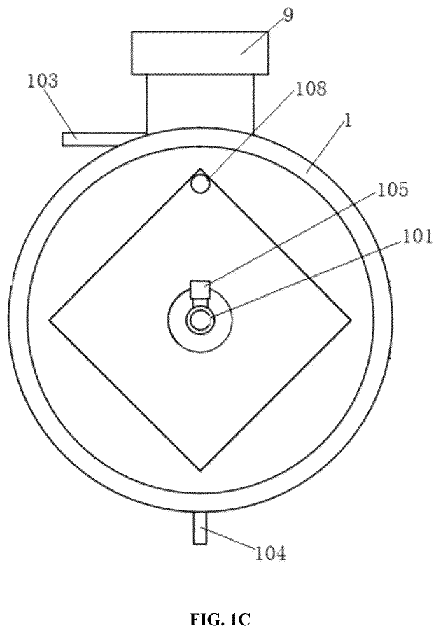

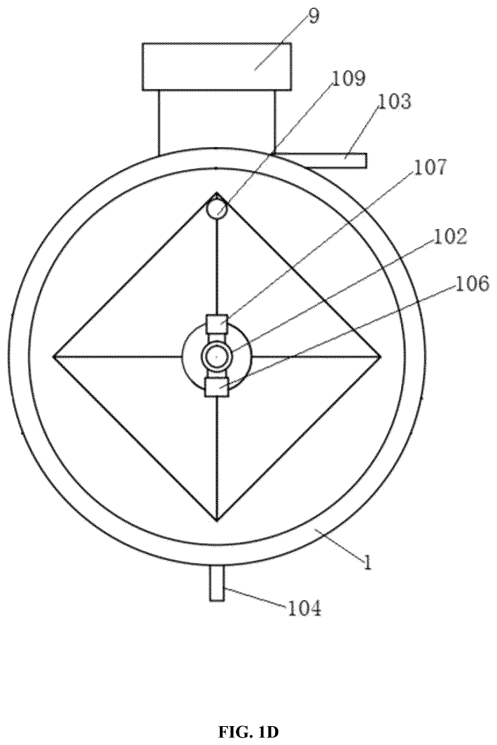

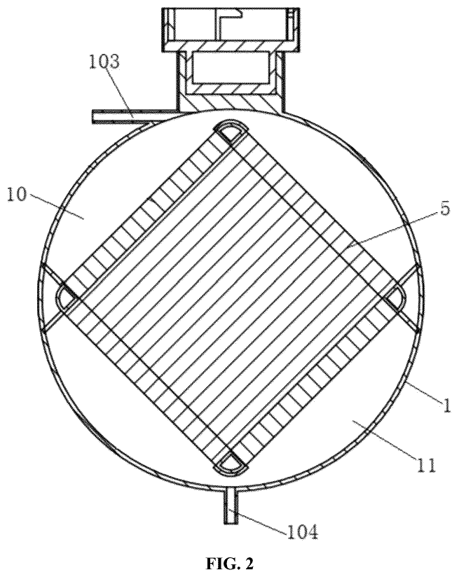

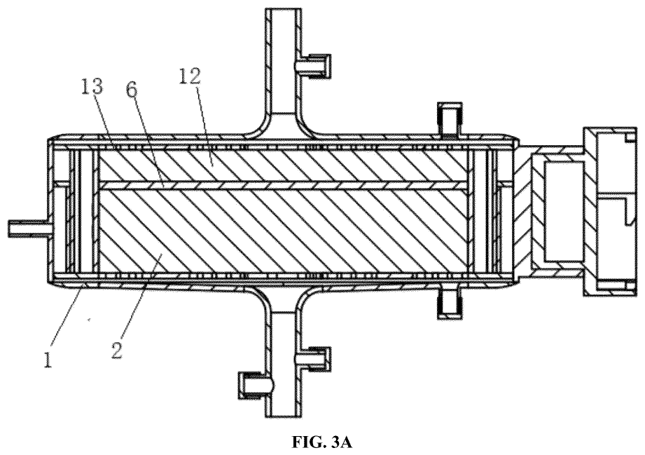

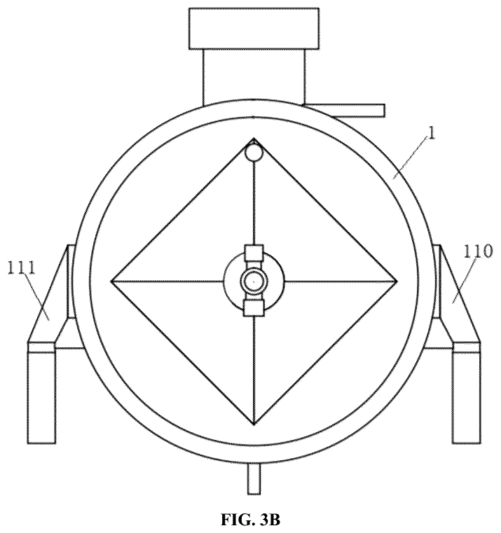

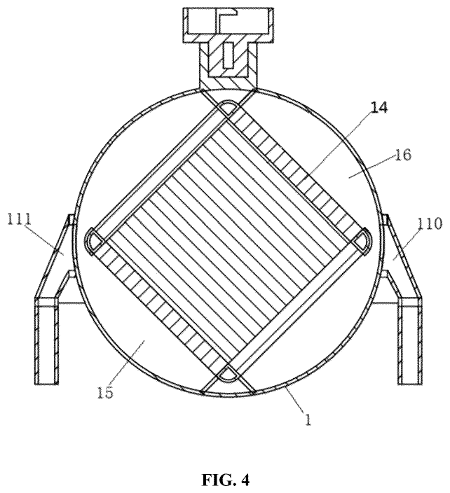

FIG. 1A is a sectional view of an oxygenator according to embodiments of the present disclosure. FIG. 1B is a partial enlarged view of a blood outlet of an oxygenator according to embodiments of the present disclosure. FIG. 1C is a top view of an oxygenator according to embodiments of the present disclosure. FIG. 1D is a bottom view of an oxygenator according to embodiments of the present disclosure.

As shown in FIG. 1, it is provided an oxygenator according to the embodiments of the present disclosure. The oxygenator includes:
a housing 1, where an upper end cover 112 of the housing 1 is provided with a blood inlet 101, and a lower end cover 113 of the housing 1 is provided with a blood outlet 102; and
an oxygenation chamber 2 arranged in the housing 1, where the axis of the blood inlet 101 and the axis of the blood outlet 102 coincide with the axis of the oxygenation chamber 2.

In this embodiment, the blood inlet 101 and the blood outlet 102 are arranged at the center of the upper and lower parts of the oxygenation chamber 2, such that after the blood uniformly diffused to the periphery in an inlet buffer zone, the blood uniformly flows to the blood outlet 102 from top to bottom due to gravity. The blood flow resistance is low, the gas-blood exchange is sufficient, and the blood retention time is short. Moreover, during the blood flow, flow fields and pressure fields in the oxygenation chamber 2 are uniformly distributed, flow dead zones or flow disturbance zones can be eliminated, thereby reducing the probability of thrombosis.

In some embodiments, an interval space 3 and an interval space 4 are formed between the upper end cover 112 of the housing 1 and the upper end face 201 of the oxygenation and between the lower end cover 113 of the housing 1 and the lower end face 202 of the oxygenation chamber, respectively. That is, the interval space 3 is formed between the upper end cover 112 of the housing 1 and the upper end face 201 of the oxygenation chamber 2, the interval space 4 is formed between the lower end cover 113 of the housing and the lower end face 202 of the oxygenation chamber 2. Spaces of the blood inlet 101 and the blood outlet 102 are gradually reduced in directions away from the interval space 3 and the interval space 4, respectively.

In this embodiment, the interval space 3 located on the upper end face 201 of the oxygenation chamber 2 can make the blood diffused prior to entering the oxygenation chamber 2 without the interference of a gas-blood exchange module, the diffusion effect is better, and the diffusion efficiency is higher. The interval space 4 located on the lower end face 202 of the oxygenation chamber 2 is used to lengthen a circulation path of blood, such that the blood can flow through this interval space 4 before flowing out of the blood outlet 102, and a possible dead zone is set outside the oxygenation chamber 2, thereby further reducing the probability of thrombosis.

In some embodiments, an inner surface of the lower end cover 113 of the housing 1 is a tapered face, and the blood outlet 102 is provided at the lowest point of the tapered face.

In this embodiment, the inner surface of the lower end cover 113 of the housing 1 is designed as a tapered face, which can pool the blood from the periphery of the interval space 4 to the blood outlet 102, thus preventing the flow dead zone from occurring in the interval space 4 to affect the blood circulation performance.

In some embodiments, the upper end face 201 and the lower end face 202 of the oxygenation chamber 2 are respectively provided with a first orifice plate 6 and a second orifice plate 7 for respectively communicating the oxygenation chamber 2 with the interval space 3 and communicating the oxygenation chamber 2 with the interval space 4.

In this embodiment, the first orifice plate 6 and the second orifice plate 7 not only can be used to fix a gas-blood exchange module in the oxygenation chamber 2, but also can improve the blood diffusion effect through holes uniformly provided on the orifice plates. In addition, the second orifice plate 7 may also be used to drain the blood at the periphery in the oxygenation chamber 2, so as to reduce the occurrence of the dead zone.

In some embodiments, a first gas exchange cavity 10 and a second gas exchange cavity 11 are formed between a side face of the oxygenation chamber 2 and a side face of the housing 1, and are used to exchange gas with the outside of the housing 1.

In this embodiment, the first gas exchange cavity 10 and the second gas exchange cavity 11 may directly carry out gas-blood exchange with the blood in the oxygenation chamber 2 without additionally providing a gas-blood exchange module. For example, the first gas exchange cavity 10 and the second gas exchange cavity 11 exchange gas with the outside of the housing 1 through gas holes 103 and 104 on the housing 1. The first gas exchange cavity 10 and the second gas exchange cavity 11 carry out gas-blood exchange with the blood in the oxygenation chamber 2 through side faces connected to the oxygenation chamber 2.

In some embodiments, multiple hollow permeable tubes 5 are arranged in the oxygenation chamber 2 in a horizontal direction. One end of each of the hollow permeable tubes 5 communicates with the first gas exchange cavity 10, and the other end of the hollow permeable tube 5 communicates with the second gas exchange cavity 11.

In this embodiment, the hollow permeable tubes 5 are arranged in the oxygenation chamber 2. Both ends of the hollow permeable tubes 5 are respectively connected to the first gas exchange cavity 10 and the second gas exchange cavity 11. The first gas exchange cavity 10 and the second gas exchange cavity 11 exchange gas with the outside to make the gas in the hollow permeable tubes 5 flow, thereby carrying out gas-blood exchange with the blood in the oxygenation chamber 2. Although a gas-blood exchange module is additionally provided, the hollow permeable tubes 5 can be uniformly distributed inside the oxygenation chamber 2, and better gas-blood exchange efficiency can be achieved. In this embodiment, the hollow permeable tubes 5 may be hollow fibrous membrane fibers.

In some embodiments, the multiple hollow permeable tubes 5 are arranged in a layered and crossed manner.

In this embodiment, the hollow permeable tubes 5 are horizontally arranged in a layered and crossed manner, such that a gas-blood exchange path can be divided into two paths from one. With the characteristic that the blood is diffused to the periphery in this embodiment, the gas-blood exchange efficiency of the membrane fibers can be greatly improved, and the efficacy of the membrane oxygenator is further improved.

In some embodiments, the oxygenator further includes:
a heat exchange chamber 12 arranged in the housing 1, where a lower end face of the heat exchange chamber 12 is connected to the upper end face 201 of the oxygenation chamber 2, and the axis of the heat exchange chamber 12 coincides with the axis of the oxygenation chamber 2.

In this embodiment, the oxygenator is additionally provided with the heat exchange chamber 12 on the basis of the foregoing embodiments, thus making the oxygenator integrate a heat exchange function without changing the original function.

In some embodiments, an upper end face of the heat exchange chamber 12 is provided with a third orifice plate 13.

In this embodiment, the original first orifice plate 16 is cancelled, or the original first orifice plate 6 is used to communicate the heat exchange chamber 12 with the oxygenation chamber 2, and the third orifice plate 13 is used to communicate the heat exchange chamber 12 with the blood inlet 101 or the interval space 3, and also has the function of improving the blood diffusion effect.

In some embodiments, a first heat exchange cavity 15 and a second heat exchange cavity 16 are formed between a side face of the heat exchange chamber 12 and the side face of the housing 1, and are connected to the outside of the housing 1 for heat exchange.

In this embodiment, the first heat exchange cavity 15 and the second heat exchange cavity 16 may directly exchange heat with the blood in the heat exchange chamber 12 without additionally providing a gas-heat exchange module. For example, the first heat exchange cavity 15 and the second heat exchange cavity 16 exchange heat with the outside of the housing 1 through heat exchange holes 110 and 111 on the housing, and the first heat exchange cavity 15 and the second heat exchange cavity 16 exchange heat with the blood in the heat exchange chamber through side faces connected to the heat exchange chamber 12.

In some embodiments, multiple heat exchange tubes 14 are arranged in the heat exchange chamber 12 in a horizontal direction. One end of each of the heat exchange tubes 14 communicates with the first heat exchange cavity 15, and the other end of the heat exchange tube 14 communicates with the second heat exchange cavity 16.

In this embodiment, the heat exchange tubes 14 are arranged in the heat exchange chamber 12. Both ends of the heat exchange tubes 14 are respectively connected to the first heat exchange cavity 15 and the second heat exchange cavity 16. The first heat exchange cavity 15 and the second heat exchange cavity 16 exchange heat with the outside to make heat media flow in the heat exchange tubes 14, thereby exchanging heat with the blood in the heat exchange chamber 12. Although a heat exchange module is additionally provided, the heat exchange tubes 14 can be uniformly distributed inside the heat exchange chamber 12, and better heat exchange efficiency can be achieved. In this embodiment, the heat exchange tubes 14 may be plastic fibers.

In some embodiments, the multiple heat exchange tubes 14 are arranged in a layered and crossed manner.

In this embodiment, as the heat exchange tubes 14 are horizontally arranged in a layered and crossed manner, a heat exchange path is divided into two paths from one. With the characteristic that the blood is diffused to the periphery in this embodiment, the heat exchange efficiency can be greatly improved, and the efficacy of the membrane oxygenator is further improved.

In some embodiments, the blood inlet 101 is provided with a first blood sampling port 105, and the blood outlet 102 is provided with a temperature measurement port 106 and a second blood sampling port 107. The first blood sampling port 105 is used for sampling and detecting the blood prior to oxygenation, the second blood sampling ort 107 is used for sampling and detecting the blood after oxygenation, and the temperature measurement port 106 is used for detecting a temperature of the blood after oxygenation in real time, thus determining whether the blood after oxygenation can directly flow into arteries and veins.

In some embodiments, the housing 1 is further provided with a mounting block 8, and a connector 9 connected to a blood storage tank is mounted on the mounting block 8.

In an embodiment, the housing 1 is further provided with a first exhaust port 108, and the first exhaust port 108 communicates with the interval space 3. The housing 1 is further provided with a second exhaust port 109, and the second exhaust port 109 communicates with the interval space 4. The first exhaust port 108 and the second exhaust port 109 are used to remove bubbles from the blood. It is also provided an extracorporeal membrane oxygenation device according to the embodiments of the present disclosure, which includes the oxygenator of any one of the foregoing embodiments.

The extracorporeal membrane oxygenation device of the embodiment has the advantages of the oxygenator of any one of the foregoing embodiments, and thus will not be described in detail here.

It should be understood that the above specific embodiments of the present disclosure are only used to illustrate or explain the principles of the present disclosure, and do not constitute limitations on the present disclosure. Therefore, any modification, equivalent substitution, improvement, etc. made without departing from the spirit and scope of the present disclosure should be included in the scope of protection of the present disclosure. Furthermore, the appended claims of the present disclosure are intended to cover all changes and modifications that come within the scope and boundaries of the appended claims, or the equivalents of such scope and boundaries.

1. An oxygenator, comprising:
a housing, an upper end cover of the housing being provided with a blood inlet, and a lower end cover of the housing being provided with a blood outlet; and
an oxygenation chamber, arranged in the housing, wherein an axis of the blood inlet and an axis of the blood outlet coincide with the axis of the oxygenation chamber.

2. The oxygenator according to claim 1, wherein
an interval space is formed between the upper end cover of the housing and an upper end face of the oxygenation chamber, and an interval space is formed between the lower end cover of the housing and a lower end face of the oxygenation chamber, and spaces of the blood inlet and the blood outlet are gradually reduced in directions away from the interval space and the interval space, respectively.

3. The oxygenator according to claim 2, wherein
an inner surface of the lower end cover of the housing is a tapered face, and the blood outlet is provided at a lowest point of the tapered face.

4. The oxygenator according to claim 2, wherein
the upper end face and the lower end face of the oxygenation chamber are respectively provided with a first orifice plate and a second orifice plate for respectively communicating the oxygenation chamber with the interval space and communicating the oxygenation chamber with the interval space.

5. The oxygenator according to claim 4, wherein
a first gas exchange cavity and a second gas exchange cavity are formed between a side face of the oxygenation chamber and a side face of the housing and are used to exchange gas with an outside of the housing.

6. The oxygenator according to claim 5, wherein
a plurality of hollow permeable tubes are arranged in the oxygenation chamber in a horizontal direction, one end of each of the hollow permeable tubes communicates with the first gas exchange cavity, and the other end of the hollow permeable tube communicates with the second gas exchange cavity.

7. The oxygenator according to claim 6, wherein
the plurality of hollow permeable tubes are arranged in a layered and crossed manner.

8. The oxygenator according to claim 1, wherein the oxygenator further comprises:
a heat exchange chamber, arranged in the housing, wherein a lower end face of the heat exchange chamber is connected to an upper end face of the oxygenation chamber, and an axis of the heat exchange chamber coincides with an axis of the oxygenation chamber.

9. The oxygenator according to claim 8, wherein
an upper end face of the heat exchange chamber is provided with a third orifice plate.

10. The oxygenator according to claim 9, wherein
a first heat exchange cavity and a second heat exchange cavity are formed between a side face of the heat exchange chamber and the side face of the housing, and are connected to an outside of the housing for heat exchange.

11. The oxygenator according to claim 10, wherein
a plurality of heat exchange tubes are arranged in the heat exchange chamber in a horizontal direction, one end of each of the heat exchange tubes communicates with the first heat exchange cavity, and the other end of the heat exchange tube communicates with the second heat exchange cavity.

12. The oxygenator according to claim 11, wherein
the plurality of heat exchange tubes are arranged in a layered and crossed manner.

13. An extracorporeal membrane oxygenation device, comprising:
an oxygenator, comprising:
a housing, an upper end cover of the housing being provided with a blood inlet, and a lower end cover of the housing being provided with a blood outlet; and
an oxygenation chamber, arranged in the housing, wherein an axis of the blood inlet and an axis of the blood outlet coincide with the axis of the oxygenation chamber.

14. The extracorporeal membrane oxygenation device according to claim 13, wherein
an interval space is formed between the upper end cover of the housing and an upper end face of the oxygenation chamber, and an interval space is formed between the lower end cover of the housing and a lower end face of the oxygenation chamber, and spaces of the blood inlet and the blood outlet are gradually reduced in directions away from the interval space and the interval space, respectively.

15. The extracorporeal membrane oxygenation device according to claim 14, wherein
an inner surface of the lower end cover of the housing is a tapered face, and the blood outlet is provided at a lowest point of the tapered face.

16. The extracorporeal membrane oxygenation device according to claim 14, wherein
the upper end face and the lower end face of the oxygenation chamber are respectively provided with a first orifice plate and a second orifice plate for respectively communicating the oxygenation chamber with the interval space and communicating the oxygenation chamber with the interval space.

17. The extracorporeal membrane oxygenation device according to claim 16, wherein
a first gas exchange cavity and a second gas exchange cavity are formed between a side face of the oxygenation chamber and a side face of the housing and are used to exchange gas with an outside of the housing.

18. The extracorporeal membrane oxygenation device according to claim 17, wherein
a plurality of hollow permeable tubes are arranged in the oxygenation chamber in a horizontal direction, one end of each of the hollow permeable tubes communicates with the first gas exchange cavity, and the other end of the hollow permeable tube communicates with the second gas exchange cavity.

19. The extracorporeal membrane oxygenation device according to claim 18, wherein
the plurality of hollow permeable tubes are arranged in a layered and crossed manner.

20. The extracorporeal membrane oxygenation device according to claim 13, wherein the oxygenator further comprises:
a heat exchange chamber, arranged in the housing, wherein a lower end face of the heat exchange chamber is connected to an upper end face of the oxygenation chamber, and an axis of the heat exchange chamber coincides with an axis of the oxygenation chamber.
